(19) United States
(12) Patent Application Publication
(10) Pub. No.: US 2015/0238644 A1
(43) Pub. Date: Aug. 27, 2015

(54) HOME APPLIANCE INCLUDING PHOTOCATALYST FOR VISIBLE RAYS

(71) Applicant: LG ELECTRONICS INC., Seoul (KR)

(72) Inventors: Bongjo SUNG, Seoul (KR); Sangyoul CHAE, Busan (KR); Kyungsoo YOON, Seoul (KR)

(73) Assignee: LG Electronics Inc.

(21) Appl. No.: 14/621,127

(22) Filed: Feb. 12, 2015

(30) Foreign Application Priority Data
Feb. 26, 2014 (KR) 10-2014-0022485

(51) Int. Cl. A61L 2/08; B01J 35/00; B01J 21/06; B01J 27/18; A61L 9/18; B01J 19/12

(52) U.S. Cl. CPC A61L 2/088; A61L 9/18; B01J 19/127; B01J 21/063; B01J 27/1802; B01J 35/0006; B01J 35/004; B01J 2219/1203

(57) ABSTRACT

A home appliance is provided that may include a photocatalyst for visible rays. The home appliance may include a case that defines an exterior, and a photocatalyst disposed on an inner or outer surface of the case. The photocatalyst may react or be activated by light having a visible-ray wavelength. Silver phosphate (Ag₃PO₄) and titanium dioxide (TiO₂) may be mixed to a set weight part ratio.

### CROSS-REFERENCE TO RELATED APPLICATION

The present application claims priority under 35 U.S.C. 119 and 35 U.S.C. 365 to Korean Patent Application No. 10-2014-0022485, filed in Korea on Feb. 26, 2014, which is hereby incorporated by reference in its entirety.

### BACKGROUND

1. Field

A home appliance including a photocatalyst for visible rays is disclosed herein.

2. Background

In recent years, introduction of external gas into buildings may be minimized to reduce energy consumption. Accordingly, due to airtight buildings, indoor air pollution in the buildings is becoming more serious. As a result, various kinds of judiciary regulations with respect to indoor door pollution materials are being reinforced more and more.

While home appliances installed in homes or companies operate, indoor pollution materials may be generated and deposited within the home appliances or discharged from the home appliances. The indoor pollution materials may cause an unpleasant smell and have a bad impact on a user's health.

For example, in the case of home appliances using air containing moisture or water, such as air conditioners, dehumidifiers, air cleaners, refrigerators, or washing machines, pollution due to dust or microorganisms inside or outside the home appliances may occur.

In detail, the indoor pollution materials may be classified into (1) particle pollution materials, such as fine dust, and asbestos, for example, (2) gas pollution materials, such as voltaic organic compounds (VOC), for example, and (3) biological pollution materials, such as viruses, molds, and bacteria, for example. A photocatalyst may be used to decompose indoor pollution materials contained in air.

The photocatalyst may represent a material that receives light to accelerate a chemical reaction. The reaction using the photocatalyst is called a photochemical reaction. Examples of the photocatalyst may include metal oxide, pigment, or chlorophyll, for example. Among these, titanium dioxide (TiO₂), that is, the metal oxide is being widely used. Titanium dioxide (TiO₂) may be stable and harmless to humans.

For example, Korean Patent Registration No. 10-0615515 discloses a method for fixing a photocatalyst and a photocatalyst absorbent using the same, and more particularly, to a method for immersing titanium oxide (TiO₂) into an absorbent, such as activated carbon or carbon black, having a wide specific surface area. However, as titanium dioxide (TiO₂) reacts with UV light having a wavelength of about 385 nm or less (λ<385 nm), titanium dioxide (TiO₂) is limited in range of application. Thus, it may be difficult to utilize titanium dioxide (TiO₂) for fluorescent lights that is, used in an indoor space. In addition, related products may be degraded and reduced in mechanical property (for example, strength) due to the UV light.

In recent years, a method for modifying a titanium dioxide photocatalyst to react with visible rays having a wavelength of about 385 nm or less (λ<385 nm) has been proposed. For example, a method for doping a metal material (Mn, Ag, Cu, etc) or a non-metal material into titanium dioxide at a predetermined ratio, a method for modifying a surface of titanium dioxide itself, and a method for forming a composite with a semiconductor material have been proposed.

Particularly, Korean Patent Registration No. 10-0935512 discloses a method of manufacturing a titanium dioxide photocatalyst and the titanium dioxide photocatalyst manufactured thereby. The titanium dioxide photocatalyst, which is doped with sulfur and zirconium, exhibits catalytic activity even to visible rays and has superior surface properties to improve the catalytic activity.

Korean Patent Publication No. 10-2013-0019833 discloses a method of manufacturing an N-doped TiO₂ and N-doped TiO₂ composite visible-ray photocatalyst using urea. Metal oxide is further doped into an N-doped TiO₂, which is synthesized at room temperature and then prepared through plasticization at a high temperature to further improve catalytic activity in a visible ray region.

However, as disclosed in these patent documents, in the case of titanium dioxide, it takes a long time to modify the titanium dioxide, and also, photocatalyst efficiency is deteriorated. Thus, development of a photocatalyst capable of being used in the visible ray range and being easily prepared is required.

### BRIEF DESCRIPTION OF THE DRAWINGS

Embodiments will be described in detail with reference to the following drawings in which like reference numerals refer to like elements, and wherein:

FIGS. 1 and 2 views of a home appliance including a photocatalyst according to an embodiment;

FIG. 3 is a photograph showing a state in which the photocatalyst is applied to a predetermined surface according to an embodiment;

FIG. 4 is a view illustrating inner components of a home appliance according to another embodiment;

FIG. 5 is a view illustrating inner components of a home appliance according to still another embodiment;

FIG. 6 is a view illustrating inner components of a home appliance according to still another embodiment;

FIG. 7 is a view illustrating inner components of a home appliance according to still another embodiment;

FIGS. 8 and 9 are views of a home appliance including a photocatalyst according to another embodiment;

FIG. 10 is a view of a home appliance according to yet another embodiment;

FIG. 11 is a view of a home appliance according to yet another embodiment;

FIGS. 12 and 13 are views of a home appliance according to yet another embodiment;

FIG. 14 is a view of a home appliance according to yet another embodiment;

FIG. 15 is a view of a home appliance according to an additional embodiment;

FIGS. 16 and 17 are views of a home appliance according to an additional embodiment; and

FIG. 18 is a view of a home appliance according to an additional embodiment.

### DETAILED DESCRIPTION

Hereinafter, exemplary embodiments will be described with reference to the accompanying drawings. The embodiments may, however, be embodied in many different forms and should not be construed as being limited to the embodiments set forth herein; rather, alternate embodiments falling within the spirit and scope will fully convey the concept to those skilled in the art. Further, where possible the same or like reference numerals have been used to indicate the same or like elements, and repetitive disclosure have been omitted.

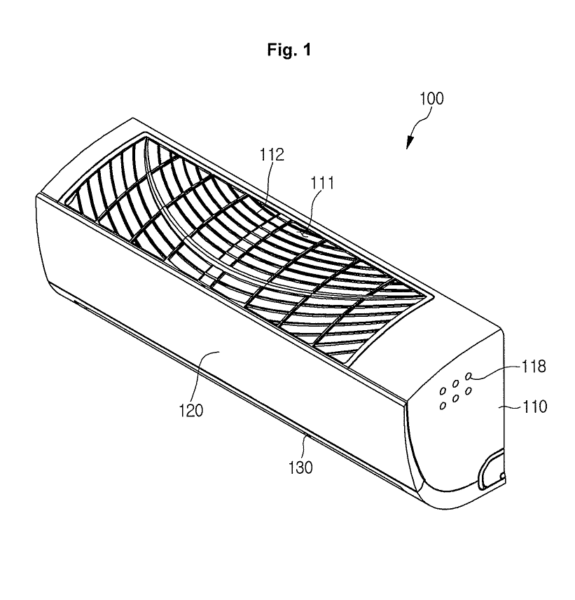

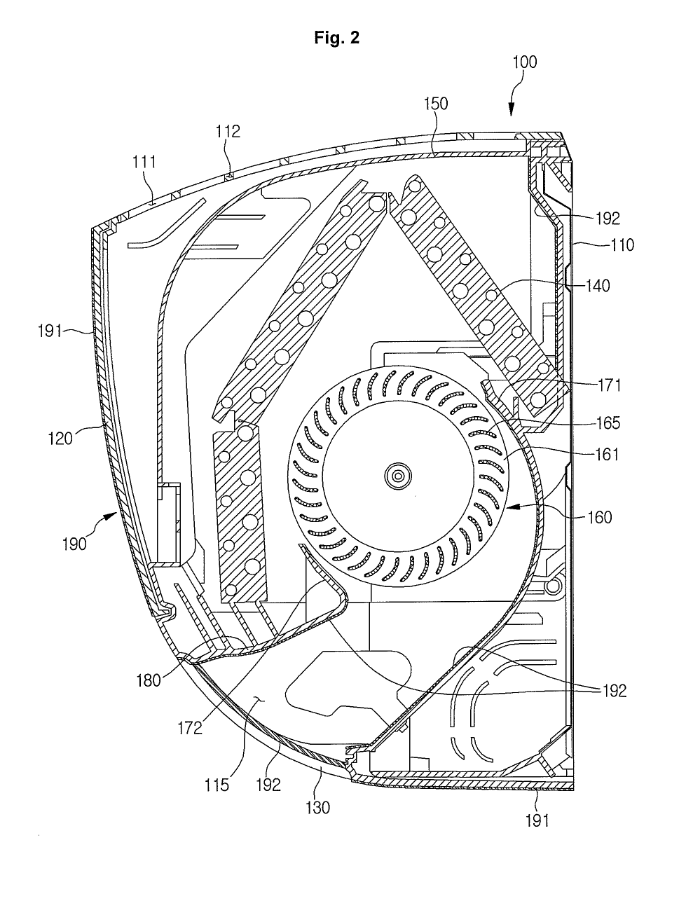

FIGS. 1 and 2 views of a home appliance including a photocatalyst part or photocatalyst according to an embodiment. Referring to FIGS. 1 and 2, a home appliance according to this embodiment may include an air conditioner 100.

The air conditioner 100 may include a case 110 that defines an exterior and accommodates a heat exchanger 140 and a fan 160 therein, and a front panel 120 coupled to a front portion of the case 110 and defining a front exterior of the air conditioner 100. The case 110 may be an indoor unit or device case disposed in an indoor space in the case of a separation type air conditioner, and a self-case of the air conditioner in the case of an integrated air conditioner. In a broad sense, the front panel 120 may be understood as one component of the case 110.

The case 110 may include a suction part or inlet 111, through which indoor air may be introduced, and a discharge part or discharge 115, through which the air introduced through the suction part 111 and heat-exchanged may be discharged into the indoor space. The suction part 111 may be formed by opening at least one portion of an upper portion of the case 110, and the discharge part 115 may be formed by opening at least one portion of a lower portion of the case 110. A suction grill 112 to prevent foreign substances from being introduced may be disposed on the suction part 111, and a discharge grill (not shown) may be disposed on the discharge part 115.

A discharge vane 130 movably disposed to open and close the discharge part 115 may be disposed on or at one side of the discharge part 115. When the discharge vane 130 is opened, air conditioned within the case 110 may be discharged into the indoor space. For example, the discharge vane 130 may be opened by rotating a lower portion of the discharge vane 130 upward.

The heat exchanger 140 that performs heat-exchange with the air suctioned through the suction part 111 may be disposed in the case 110. The heat exchanger 140 may include a refrigerant tube, through which a refrigerant may flow, and a heat exchange fin coupled to the refrigerant tube to increase a heat exchange area. The heat exchanger 140 may be disposed to surround a suction-side of the fan 160. For example, the heat exchanger 140 may include a plurality of heat exchange parts or portions that are bent.

The fan 160 may include a cross-flow fan to radially discharge the air suctioned in a circumferential direction. The fan 160 may include a fan body 161, and a plurality of blades 165 fixed to one side of the fan body 161 and disposed to be spaced apart from each other in the circumferential direction. That is, the plurality of blades 165 of the fan 160 may be arranged along the circumferential direction.

Passage guides 171 and 172 disposed in a vicinity of an outer circumferential surface of the fan 160 to guide a flow of the air may be disposed in the case 110. The passage guides 171 and 172 may include a rear guide 171 and a stabilizer 172.

The rear guide 171 may extend from a rear portion of the case 10 toward a suction-side of the fan 160. The rear guide 171 may guide the suctioned air to allow the suctioned air to smoothly flow toward the fan 160 when the fan 160 rotates. The rear guide 171 may also prevent the air flowing by the fan 160 from being delaminated in the fan 160.

The stabilizer 172 may be disposed on a discharge-side of the fan 160. The stabilizer 172 may be disposed to be spaced apart from the outer circumferential surface of the fan 160 to prevent the air discharged from the fan 160 from reversely flowing toward the heat exchanger 140. The rear guide 171 and the stabilizer 172 may extend along a longitudinal direction of the fan 160.

A drain part or drain 180 to store condensate water generated while the air and refrigerant are heat-exchanged with each other may be disposed under the heat exchanger 140. Further, a filter 150 to filter foreign substances contained in the air suctioned through the suction part 111 may be disposed in the case 110. The filter 150 may be disposed inside the suction part 111 to surround the heat exchanger 140. The air filtered by the filter 150 may flow toward the heat exchanger 140.

A photocatalyst part or photocatalyst 190 that serves as a photocatalyst that reacts or is activated by visible rays may be disposed inside or outside of the case 110. The photocatalyst part 190 may include a first photocatalyst part or photocatalyst 191 disposed on an outer surface of the case 110. The first photocatalyst part 191 may be disposed on the outer surface of the case 110 in addition to the front panel 120. The photocatalyst part 190 further include a second photocatalyst part or photocatalyst 192 disposed on an inner surface of the case 110. The second photocatalyst part 192 may be disposed the inner surface of the case 110 and a surface of a component disposed in the case 110, that is, a surface of each of the passage guides 171 and 172 or the discharge vane 130.

One or more guide hole or guide groove 118 that serves as a “guide unit” a “guide” to transmit light emitted from the outside of the case 110 (hereinafter, referred to as “external light”) into the case 110 may be defined in the case 110. The guide hole(s) 118 may pass through at least a portion of the case 110. The guide hole(s) 118 may be defined in the case 110. A cover member formed of a transparent material capable of transmitting the external light therethrough may be disposed on the guide groove.

The external light may include natural light existing in the indoor space in which the air conditioner is installed or light emitted from a light source disposed in the indoor space, for example, a fluorescent light, an incandescent light, or an LED. Light emitted from an external light source may be induced into the case 110 through the guide hole(s) 118 to activate the second photocatalyst part 192. Of course, the first photocatalyst part 191 disposed on the outer surface of the case 110 may directly react or be activated by the external light.

Hereinafter, the photocatalyst part 190 will be described with reference to the accompanying drawings.

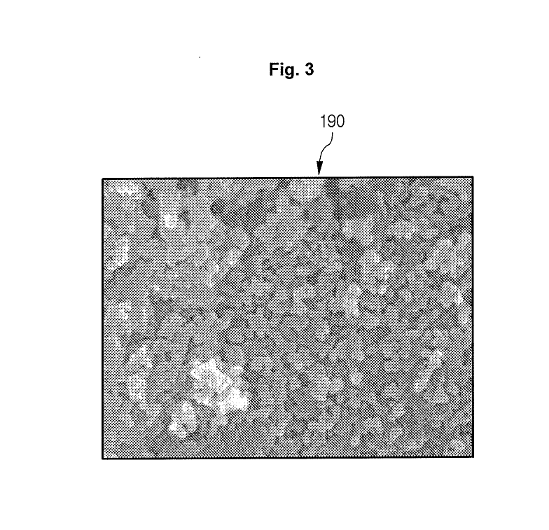

FIG. 3 is a photograph showing a state in which the photocatalyst part is applied to a predetermined surface according to this embodiment. Referring to FIG. 3, the photocatalyst part 190 according to this embodiment may include a plurality of composites. In detail, the plurality of composites may include silver phosphate (Ag₃PO₄), titanium dioxide (TiO₂), and inorganic binder. For example, the plurality of composites may include about 20 parts to about 50 parts by weight of silver phosphate (Ag₃PO₄), about 5 parts to about 40 parts by weight of titanium dioxide (TiO₂), and about 10 parts to about 40 parts by weight of inorganic binder.

The photocatalyst part 190 may be provided in the form of a solution in which the plurality of composites are mixed with a predetermined solvent. The photocatalyst part 190 may be bonded to the outer or inner surface of the case 110. The photocatalyst part 190 may be bonded to the case 110 through, for example, coating. For example, the coating may include dip coating, spray coating, or screen printing, for example. In the case of dip coating, a drying temperature may vary according to characteristics of a base material for coating. For example, the dip coating may be performed at a temperature of about 148° C. to about 152° C. for about 9 minutes to about 11 minutes.

As described above, the photocatalyst part 190 may be prepared in the form of the solution and applied to the case 110. Thus, the photocatalyst part 190 may be easily bonded to the surface of the case 110 (bonding force securement).

The plurality of composites will be described below in detail.

(1) Silver Phosphate (Ag₃PO₄)

Silver phosphate (Ag₃PO₄) may be a material that has significantly high oxidizing power in the visible ray region. The composites for coating may contain about 20 parts to about 50 parts by weight of silver phosphate (Ag₃PO₄). If the silver phosphate (Ag₃PO₄) has about 20 parts by weight or less, catalytic activity may be low. On the other hand, if the silver phosphate (Ag₃PO₄) has about 50 parts by weight or more, a cost-performance ratio may be high, and also, dispersibility may be deteriorated to cause aggregation, thereby reducing a specific surface area.

The silver phosphate (Ag₃PO₄) may be prepared through ionic exchange reaction between silver chloride (AgCl) and sodium phosphate (NaPO₄). Also, to increase a specific surface area when the silver phosphate (Ag₃PO₄) is applied to the photocatalyst, the silver phosphate (Ag₃PO₄) may range in size from nanometers to micrometers.

For example, the silver phosphate (Ag₃PO₄) may be used in the form of powder having a mean diameter of about 20 nm to about 50 nm. Alternatively, the silver phosphate (Ag₃PO₄) may be used in a state in which the silver phosphate (Ag₃PO₄) is synthesized in a liquid phase by using a solvent and then dispersed into the solvent. A material capable of dissolving the silver phosphate (Ag₃PO₄) may be used as the solvent. A mixed solution with a ratio of water:ethanol=3:4 may be used as the solvent. The silver phosphate (Ag₃PO₄) synthesized in the liquid phase may have a mean particle size of about 20 nm to about 50 nm.

As the silver phosphate (Ag₃PO₄) has a uniform and fine particle size to increase a specific surface area (unit, m²/g) of a material, adhesion to a basic material for coating may increase to enhance catalytic activity. As the silver phosphate (Ag₃PO₄) has significantly high oxidizing power in the visible ray region, oxygen generation (unit, μmol) may be high, and decomposition of methylene blue (MB), which is a blue dye, is superior when compared to a general photocatalyst (TiO₂, WO₃, BiVO₄, etc). Also, the silver phosphate (Ag₃PO₄), which requires optical energy having a visible-ray wavelength range of about 385 nm or more and a mean wavelength of about 500 nm, may easily cause the catalytic activity reaction in general light.

The silver phosphate (Ag₃PO₄) in itself may have antibacterial (bacteria, and mold, for example) performance and a synergy effect, such as, decomposition efficiency of organic materials (microorganism, and bad small component, for example) through simultaneous activity with titanium dioxide (TiO₂) in low energy (the visible-ray wavelength range) by the silver phosphate (Ag₃PO₄).

(2) Titanium Dioxide (TiO₂)

Titanium dioxide (TiO₂) may be a representative photocatalyst material that exhibits high activity when UV rays are irradiated and is chemically stable without being eroded by an acid, a base, and an organic solvent. The plurality of composites may contain about 5 parts to about 40 parts by weight of titanium dioxide (TiO₂). If the titanium dioxide (TiO₂) has a content less than the above-described content, visible-ray catalyst performance may not be sufficiently exhibited, as if the silver phosphate (Ag₃PO₄) is used solely, to deteriorate overall catalytic activity. On the other hand, if the dioxide (TiO₂) has a content greater than the above-described content, as a ratio of titanium dioxide (TiO₂) that serves as a catalytic activity assistant is too high, the photocatalytic activity may be deteriorated.

The titanium dioxide (TiO₂) may be synthesized using TiCl4 as a precursor. In this case, a crystal structure of the titanium dioxide (TiO₂) may have an anatase shape.

The titanium dioxide (TiO₂) may be used in the form of powder having a mean diameter of about 20 nm to about 25 nm. Alternatively, the titanium dioxide (TiO₂) may be used in a state in which the titanium dioxide (TiO₂) is synthesized in a liquid phase by using a solvent and then dispersed into the solvent. A material capable of dissolving the titanium dioxide (TiO₂) may be used as the solvent. A mixed solution with a ratio of water:ethanol=3:4 may be used as the solvent. The titanium dioxide (TiO₂) synthesized in the liquid phase may have a mean particle size of about 20 nm to about 25 nm.

(3) Inorganic Binder

The plurality of composites may include an inorganic binder. The inorganic binder may include a polysilicate compound. The polysilicate compound may be composed of colloidal silica (SiO₂) and metal alkoxide.

The colloidal silica (SiO₂) may have a size of about 20 nm. Also, the metal alkoxide may be selected from a group consisting of silicon alkoxide ((C₂H₅O)₄Si, C₉H₂₀O₅Si), titanium alkoxide, zirconium alkoxide, aluminum alkoxide, and a combination thereof.

The inorganic binder may be used in a state in which the inorganic binder is dispersed into a mixture with a ratio of water (H₂O):ethanol (C₂H₅OH)=3:4 as a solvent. The inorganic binder may contain about 10 parts to about 30 parts by weight of colloidal silica (SiO₂). If the inorganic binder has a content less than the above-described content, a function as the binder may not be properly performed to cause separation between other compounds and a basic material. On the other hand, if the inorganic binder has a content greater than the above-described content, the photocatalyst particles may be covered by the binder, deteriorating catalytic activity.

The inorganic binder may contain about 3 parts to about 10 parts by weight of metal alkoxide ((C₂H₅O)₄Si) and about 10 parts to about 30 parts by weight of metal alkoxide (C₉H₂₀O₅Si). If the content of the inorganic binder is beyond the above-described contents, adhesion to a basic material for coating may be reduced, and thus, the coating may not be performed.

The inorganic binder may include additional other components. The other components may be selected by a person skilled in the art in consideration of a final composition for coating. For example, the inorganic binder may include a stabilizer, an acid catalyst, a hardener, and/or a metal additive, for example.

The stabilizer may be selected from a group consisting of acetyl acetone, ethyl acetoacetate, iron acetoacetate, alkanolamine, and a combination thereof. The inorganic binder may contain about 0.1 parts to about 0.5 parts by weight of stabilizer.

The acid catalyst may be selected from a group consisting of a phosphate metal catalyst, a nitrate metal catalyst, a phosphate-chloride composite metal catalyst, and a combination thereof. The inorganic binder may contain about 0.01 parts to about 0.5 parts by weight of acid catalyst.

The hardener may be selected from a group consisting of aliphatic polyamine, crylonitrile-modified amine, polyaminde, amido amine, dicyandiamide, amide resin, isocyanate, melamine, and a combination thereof. The inorganic binder may contain about 0.05 parts to about 1 part by weight of hardener.

An aluminum compound may be used as the metal additive. The aluminum compound may be prepared by mixing aluminum isopropoxide with aluminum chloride. The inorganic binder may contain about 0.05 parts to about 0.5 parts by weight of metal additive.

When the photocatalyst part 190 containing the above-described composite is disposed on the surface of the case 110, water (H₂O) or oxygen (O₂) may change into reactive oxygen species (ROS) due to the catalyst effect of the photocatalyst part 190. The reactive oxygen species (ROS) may include hydroxy radical (OH—), hydrogen peroxide (H₂O₂), and the like.

The reactive oxygen species (ROS) may perform strong sterilization (oxidation) and deodorization functions. In detail, reactive oxygen species (ROS) may decompose gas pollution materials, such as toluene, and ammonia, for example, as well as biological pollution materials, such as bacteria, and molds, for example, which consist of organic materials.

In summary, as the photocatalyst part 190 may be disposed on the inner or outer surface of the case 110 according to this embodiment, generation of the pollution materials by the air or moisture, that is, accumulation of dust or propagation of microorganisms may be prevented.

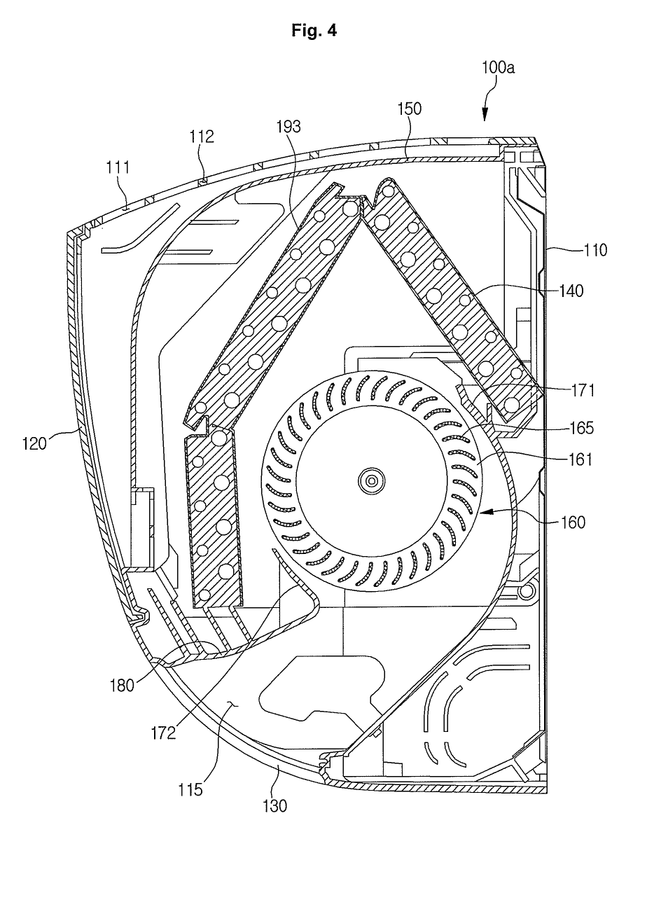

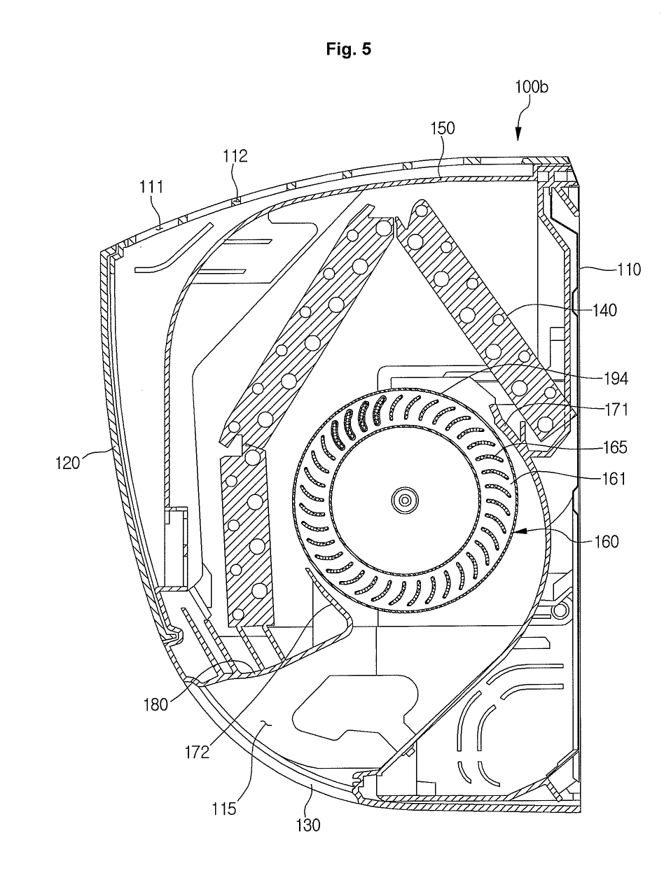

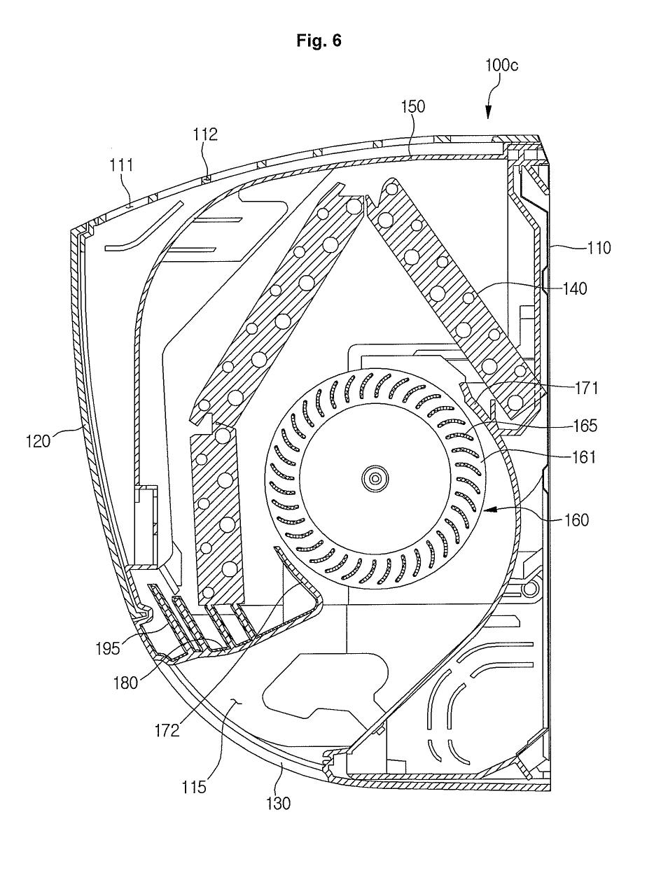

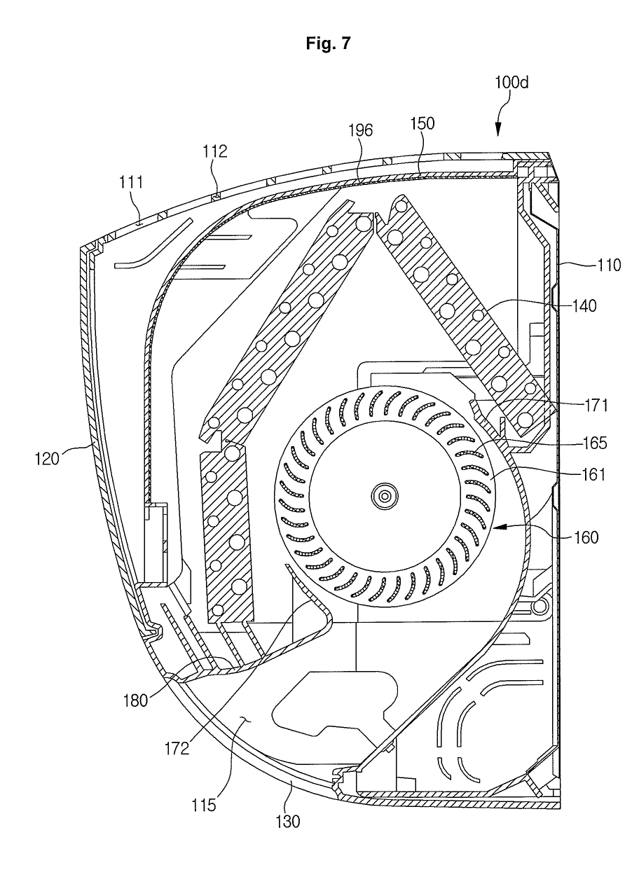

FIG. 4 is a view illustrating inner components of a home appliance according to another embodiment. FIG. 5 is a view illustrating inner components of a home appliance according to still another embodiment. FIG. 6 is a view illustrating inner components of a home appliance according to still another embodiment. FIG. 7 is a view illustrating inner components of a home appliance according to still another embodiment.

Various embodiments will be described with reference to FIGS. 4 to 7. The photocatalyst part described according to the previous embodiment may be disposed on various components disposed in the case 110, in addition to the inner or outer surface of the case 110.

In detail, reference to FIG. 4, a third photocatalyst part or photocatalyst 193 provided on or bonded to a surface of heat exchanger 140 may be disposed in case 110 of air conditioner 100a according to this embodiment. The third photocatalyst part 193 may be provided in the form of a solution and applied to the surface of the heat exchanger 140. Accordingly, a predetermined bonding force or more may be secured.

Pollution materials, such as microorganisms, may be generated on the surface of the heat exchanger 140 by condensate water generated while a refrigerant and air are heat-exchanged with each other. Thus, the third photocatalyst part 193 may be provided to restrain or prevent the generation of the pollution materials, or remove the generated pollution materials.

Referring to FIG. 5, a fourth photocatalyst part or photocatalyst 194 provided on or bonded to a surface of fan 160 may be disposed within case 110 of air conditioner 100b according to this embodiment. The fourth photocatalyst part 194 may be disposed on surfaces of fan body 161 of the fan 160 and blade 165. Also, the fourth photocatalyst part 194 may be provided in the form of a solution and applied to the surface of the fan 160. Accordingly, a predetermined bonding force or more may be secured.

Pollution materials, such as microorganisms, may be generated on the surface of the fan 160 by dust contained in flowing air or moisture contained in the air. Thus, the fourth photocatalyst part 194 may be provided to restrain or prevent the generation of the pollution materials, or remove the generated pollution materials.

Referring to FIG. 6, a fifth photocatalyst part or photocatalyst 195 provided on or bonded to a surface of drain part or drain 180 may be disposed within case 110 of air conditioner 100c according to this embodiment. The fifth photocatalyst part 195 may be provided in the form of a solution and applied to the surface of the drain part 180. Accordingly, a predetermined boding force or more may be secured.

Pollution materials, such as microorganisms, may be generated on the drain part 180 by stored condensate water. Thus, the fifth photocatalyst part 195 may be provided to restrain or prevent the generation of the pollution materials, or remove the generated pollution materials.

Referring to FIG. 7, a sixth photocatalyst part or photocatalyst 196 bonded to a surface of filter 150 may be disposed within case 110 of air conditioner 100d according to this embodiment. The sixth photocatalyst part 196 may be provided in the form of a solution and applied to the surface of the filter 150. Accordingly, a predetermined bonding force or more may be secured.

Pollution materials, such as microorganisms, may be generated on the filter 150 by dust contained in flowing air or moisture contained in the air. Thus, the sixth photocatalyst part 196 may be provided to restrain or prevent the generation of the pollution materials, or remove the generated pollution materials. Therefore, occurrence of bad smell from the filter may be prevented, and recycling and lift-cycle extension effects of the filter may be expected.

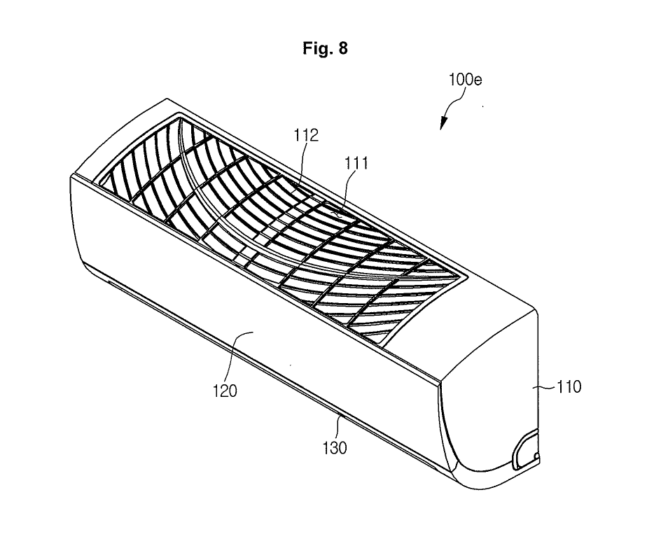

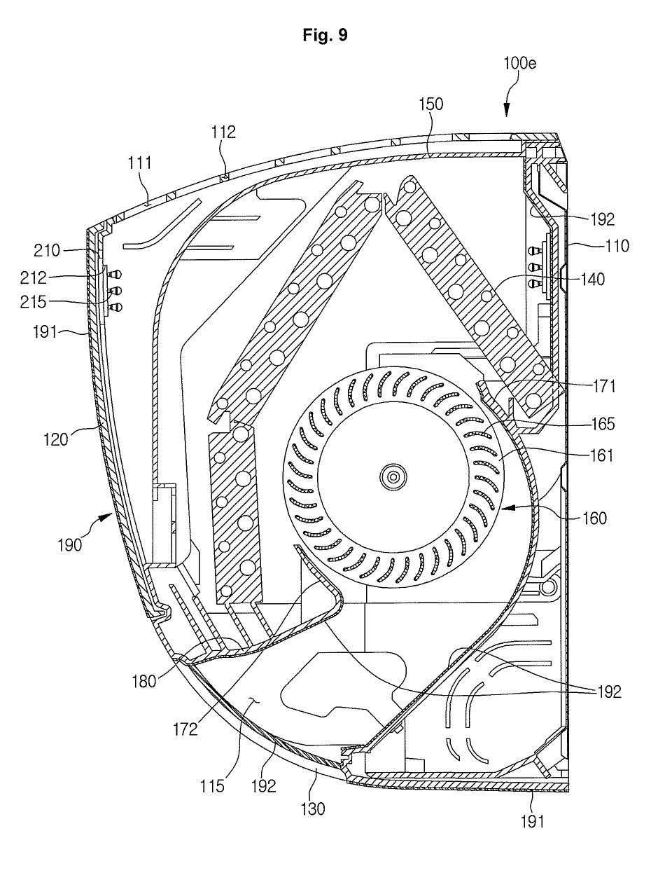

FIGS. 8 and 9 are views of a home appliance including a photocatalyst part according to a sixth embodiment. Referring to FIGS. 8 and 9, home appliance 100e according to another embodiment may include one or more lighting device 210, 212, and 215 disposed in case 110 to irradiate visible rays.

The lighting device may include a printed circuit board (PCB) 212 disposed on or at one position within the case 110, and a light source 215 that serves as a “light source”. The PCB 212 and the light source 215 may be supported on the case 110 by a light source fixing part or portion 210. The light source 215 may irradiate the visible rays onto a photocatalyst part 190. For example, the light source 215 may include a light emitting diode (LED) that emits light having a visible-ray wavelength.

A plurality of the lighting device 210, 212, and 215 may be provided. For example, the lighting device 210, 212, and 215 may be disposed on a front surface of the case 110, that is, inside front panel 120 and inside a rear surface of the case 110.

As the lighting source 215 may be provided as a visible-ray lighting source, the lighting source 215 may be used in a wide range of applications and be inexpensive when compared to a light source that irradiates light having a UV-ray wavelength. In addition, a phenomenon in which a component within the case may be degraded or weakened in strength when UV rays are repeatedly irradiated into the case 110 or irradiated into the case 110 for a long time may be prevented. As another example, the lighting source 215 may include a wire-type lighting source using an optical fiber.

Although the lighting device is disposed in case 110 including first photocatalyst part 191 and second photocatalyst part 192 in FIG. 9, embodiments are not limited thereto. For example, the lighting device may be disposed in the case 110 according to the embodiments of FIGS. 4 to 7.

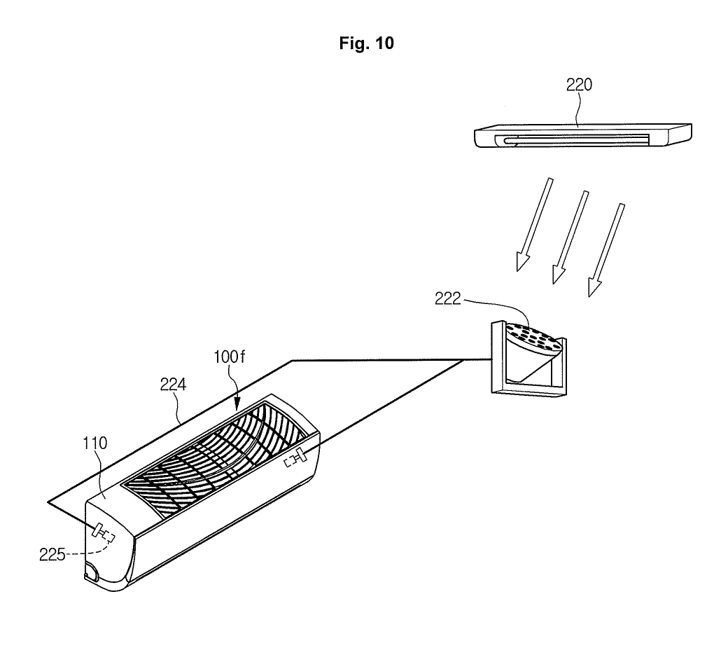

FIG. 10 is a view of a home appliance according to yet another embodiment. Referring to FIG. 10, home appliance 100f according to this embodiment may include a light source 225 disposed in case 110 and a guide device or guide 222 and 224 that guides external light emitted from an external light source 220 toward the lighting source 225. Although a lamp disposed in a building or indoor space is exemplified as the external light source 220 in FIG. 10, the light emitted from the external light source 220 may include natural light. The external light source 220 may irradiate light having a visible-ray wavelength region.

The guide device 222 and 224 may include a light collection part or portion 222 that collects light (visible rays) emitted from the external light source 220, and an optical cable 224 that guides the light collected by the light collection part 222 into the case 110, that is, toward the light source 225. The light collection part 222 may be disposed in the case 110, or at a position spaced apart from the case 110. The optical cable 224 may extend to the inside of the case 110, and then, may be coupled to the light source 225.

The light source 225 may irradiate the visible rays transmitted through the optical cable 224 into the case 110. Descriptions with respect to the light source 225 may be derived from those described with reference to FIG. 9. For example, the lighting source 225 may include a wire-type lighting source using an LED or optical fiber.

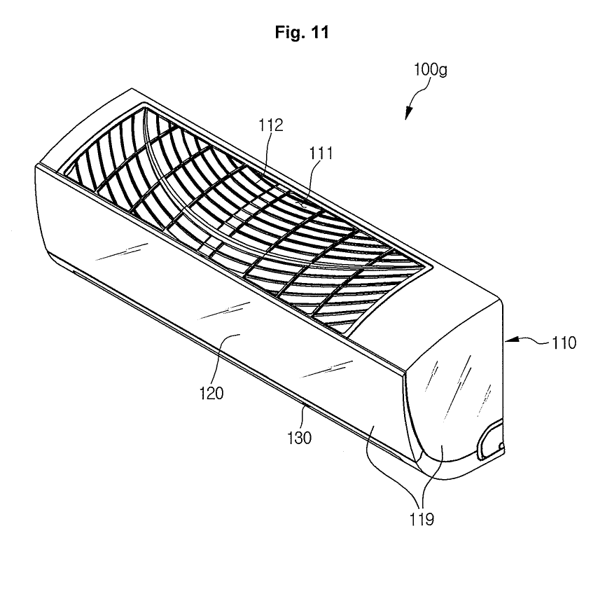

FIG. 11 is a view of a home appliance according to yet another embodiment. Referring to FIG. 11, case 110 of air conditioner 100g according to this embodiment may include a light guide part or guide 119 that guides external light into the case 110.

The light guide part 119 may be one component of the case 110. The light guide part 119 may be formed of a light-transmissive material, for example, a transparent plastic material. The light guide part 119 may be disposed on a front surface or a side surface of the case 110.

Suction part 111 may be disposed in an upper portion of the case 110 to guide the external light into the case 110. In this sense, the suction part 110 may serve as another “light guide part” or “light guide”.

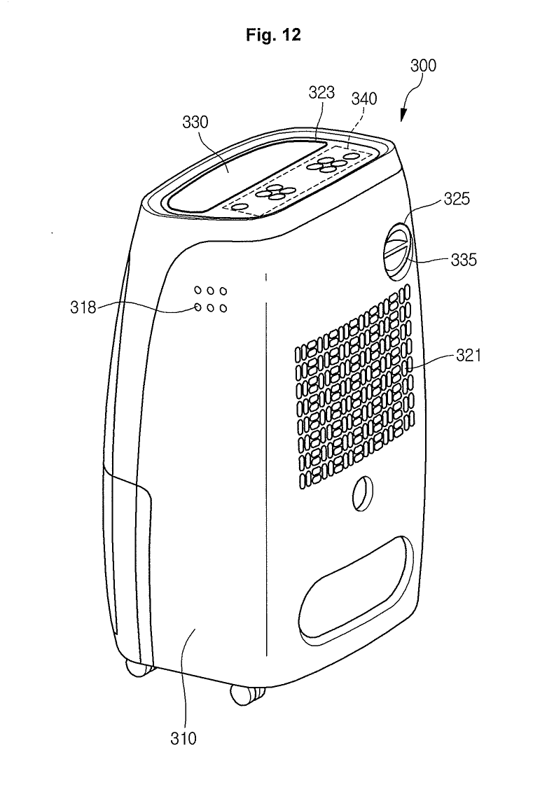

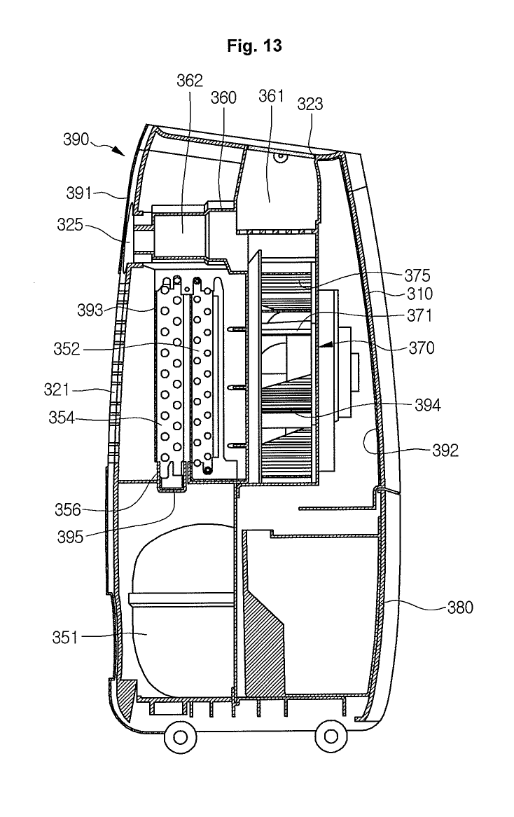

FIGS. 12 and 13 are views of a home appliance according to yet another embodiment. Referring to FIGS. 12 and 13, a home appliance according to this embodiment may include a dehumidifier 300.

The dehumidifier 300 may include a case 310 that defines an exterior thereof. The case 310 may include one or more suction hole 321, through which air may be suctioned in, and a plurality of discharge holes 323 and 325, through which air dehumidified within the case 310 may be discharged.

The plurality of discharge holes 323 and 325 may include a first discharge hole 323 and a second discharge hole 325. For example, the first discharge hole 323 may be defined in an upper portion of the case 310, and the second discharge hole 325 may be defined in a rear surface of the case 310.

The case 310 may further include a louver 330 to adjust a discharge direction of the dehumidified air discharged from the first discharge hole 323 when the first discharge hole 323 is opened. A cap 335 to open and close the second discharge hole 325 may be disposed in the case 310. The cap 335 may be separably connected to the case 310. For example, the cap 335 may rotate and then be coupled to the case 310 or separated from the case 310. In a state in which the second discharge hole 325 is opened, a predetermined hose may be connected to the second discharge hole 325. The dehumidified air discharged through the hose may be used to dry shoes.

A compressor 351, a condenser 352 that serves as a heat exchanger, and an evaporator 354 to dehumidify humid air may be disposed in the case 310. In detail, the evaporator 354 and the condenser 352 may be disposed in parallel. Air passing through the suction hole 321 may be dehumidified while passing through the evaporator 354, and then, may be heated (dried) while passing through the condenser 352. A drain part or drain 356 to store condensate water generated in the condenser 352 and the evaporator 354 may be disposed under the condenser 352 and the evaporator 354.

A fan 370 to create air flow, a discharge guide 360 to discharge the dehumidified air, and a water tank 380 to store the condensate water generated during the dehumidification may be further disposed in the case 310. The fan 370 may be a centrifugal fan. In detail, the fan 370 may include a fan body 371, and a plurality of blades 375 attached thereto.

The discharge guide 360 may include a first discharge passage 361, and a second discharge passage 362. The first discharge passage 361 may be aligned with the first discharge hole 323, and the second discharge passage 362 may be aligned with the second discharge hole 325.

A manipulation part or manipulator 340 to input a command for an operation of the dehumidifier 300 may be further disposed in or on the case 310. The manipulation part 340 may include a selection part or selector to select a dehumidification mode.

At least one photocatalyst part or photocatalyst 390 is disposed in the case 310. Descriptions with respect to the photocatalyst part 390 will be derived from those of the photocatalyst part described according to the previous embodiments.

The photocatalyst part 390 may include at least one of a first photocatalyst part or photocatalyst 391 disposed on an outer surface of the case 310, a second photocatalyst part or photocatalyst 392 disposed on an inner surface of the case 310, a third photocatalyst part or photocatalyst 393 disposed on surfaces of the condenser 352 and the evaporator 354, a fourth photocatalyst part or photocatalyst 394 disposed on a surface of the fan 370, and a fifth photocatalyst part or photocatalyst 395 disposed on the drain part 356.

One or more guide hole or groove 318 to introduce external light into the case 310 may be defined in the case 310. The guide hole(s) 318 may pass through at least a portion of the case 310. As described above, the photocatalyst part may be disposed in the dehumidifier, through which humid air or moisture may flow, to restrain or prevent generation of the pollution materials, or propagation of microorganisms.

Another embodiment will be discussed hereinbelow. Although not shown, a light guide part or guide (see the descriptions of FIG. 11) to introduce external light into case 310 may be disposed in the case 310.

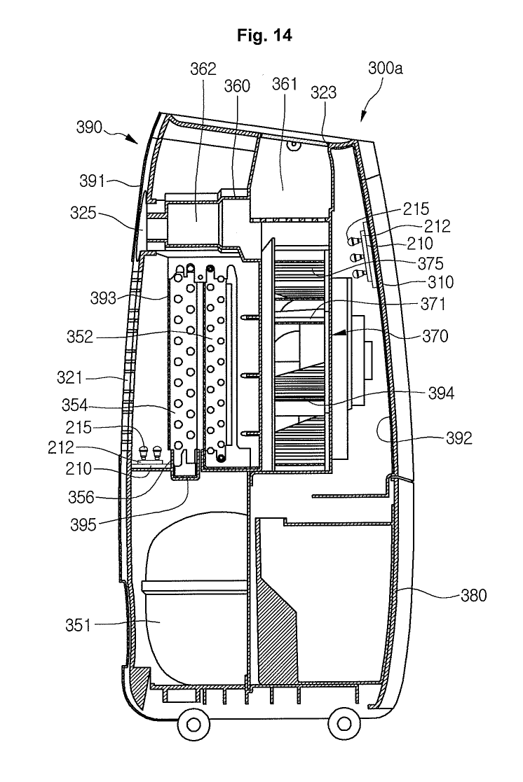

FIG. 14 is a view of a home appliance according to yet another embodiment. Referring to FIG. 14, a lighting device 210, 212, and 215 to irradiate visible rays may be disposed in case 310 of dehumidifier 300a.

The lighting device 210, 212, and 215 may include printed circuit board (PCB) 212 disposed on or at one position within the case 310, and light source 215. The PCB 212 and the light source 215 may be supported on the case 310 by light source fixing part or portion 210.

A plurality of the lighting device 210, 212, and 215 may be provided. The light source 215 may include a wire-type lighting source using an LED or optical fiber that emits light having a visible-ray wavelength.

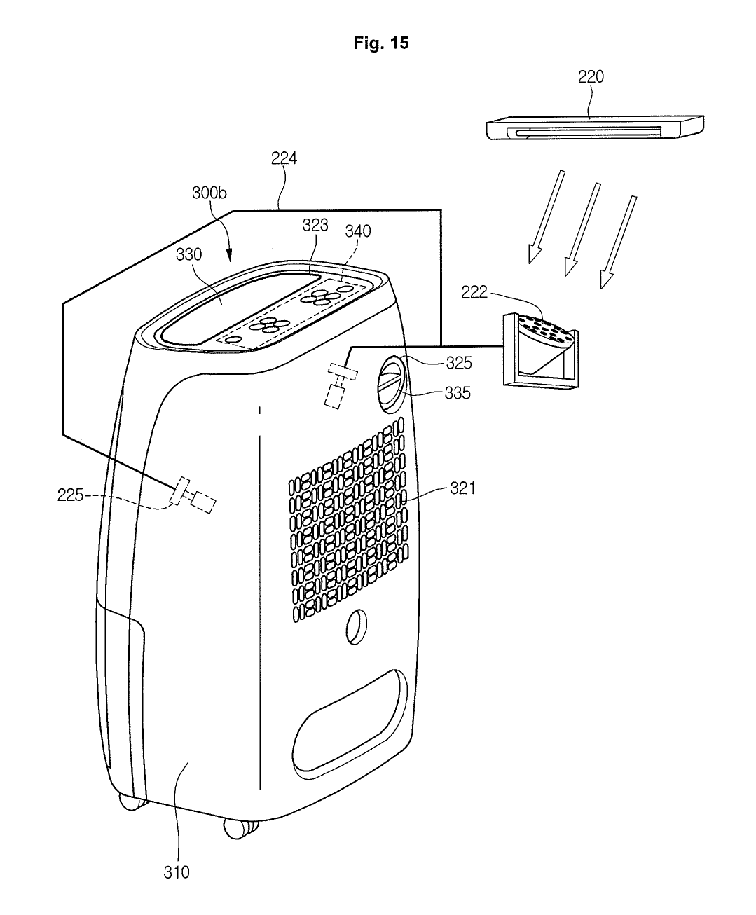

FIG. 15 is a view of a home appliance according to an additional embodiment. Referring to FIG. 15, dehumidifier 300b according to this embodiment may include light source 225 disposed in case 310 and guide device 222 and 224 to guide external light emitted from external light source 222 toward the light source 225. The external light source 220 may irradiate (or include) light having a visible-ray wavelength region.

The guide device 222 and 224 may include light collection part 222 that collects light (visible rays) emitted from the external light source 220, and optical cable 224 that guides the light collected by the light collection part 222 into the case 310. The light collection part 222 may be disposed in the case 310 or disposed at a position spaced apart from the case 310. The optical cable 224 may extend to the inside of the case 310, and then, may be coupled to the lighting source 225.

The lighting source 225 may irradiate the visible rays transmitted through the optical cable 224 into the case 310. For example, the light source 225 may include a wire-type lighting source using an LED or optical fiber.

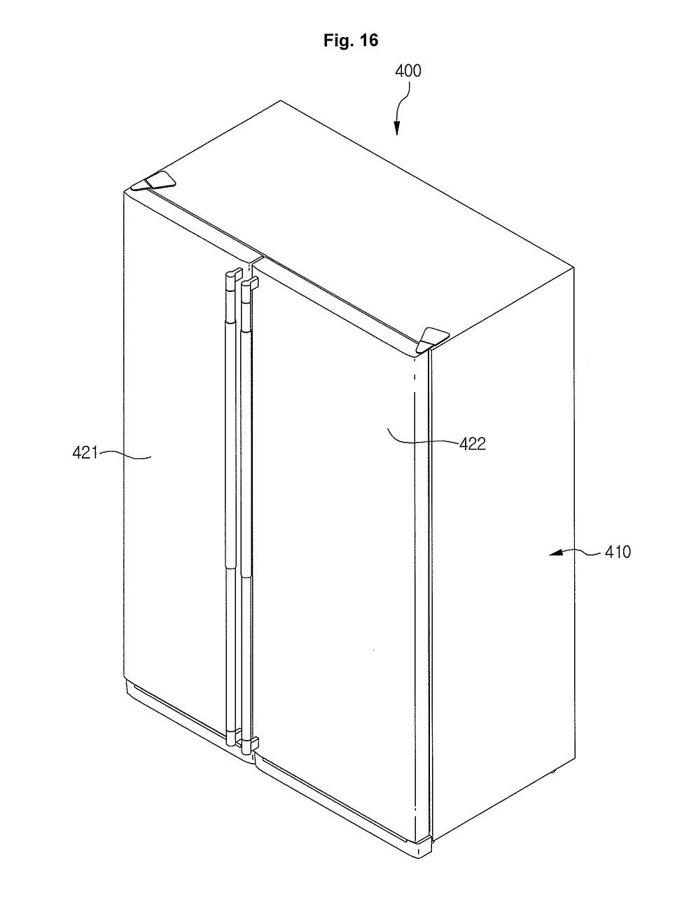

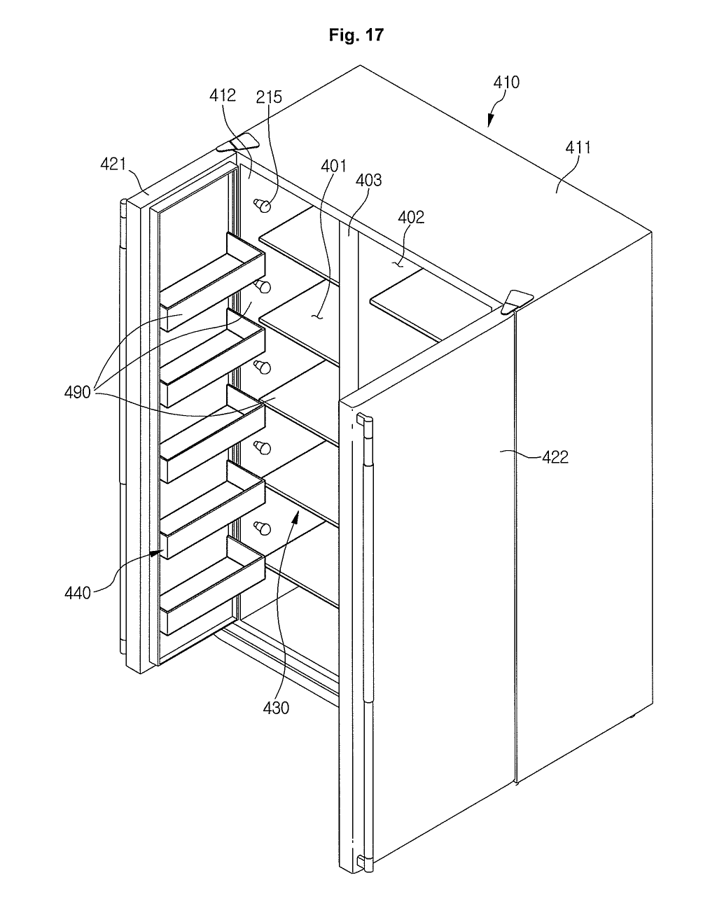

FIGS. 16 and 17 are views of a home appliance according to an additional embodiment. Referring to FIGS. 16 and 17, a home appliance according to this embodiment may include a refrigerator 400 to store food or other items in a frozen or refrigerated state.

The refrigerator 400 may include a case 410 that defines a storage compartment, and doors 421 and 422 coupled to a front portion of the case 410. The storage compartment may include a freezer compartment 401 and a refrigerator compartment 402. The freezer compartment 401 and the refrigerator compartment 402 may be partitioned by a partition wall 403.

One or more shelf 430 to accommodate food or other items may be disposed in each of the freezer compartment 401 and the refrigerator compartment 402. A plurality of the shelf 430 may be provided. The plurality of shelves 430 may be disposed to be vertically spaced apart from each other. The freezer compartment 401 and/or the refrigerator compartment 402 may be partitioned into a plurality of spaces by the plurality of shelves 430.

The case 410 may include an outer case 411 that defines an exterior of the refrigerator 400, and an inner case 412 that defines an interior of the refrigerator 400. The outer case 411 and the inner case 412 may be coupled to each other, and an insulation material (not shown) may be disposed between the outer case 411 and the inner case 412. One or more basket 440 to accommodate food or other items may be disposed on a back surface of each of the doors 421 and 422. A plurality of the basket 440 may be provided. The plurality of baskets 440 may be disposed to be spaced apart from each other.

A photocatalyst part or photocatalyst 490 may be disposed in the case 410. Descriptions with respect to the photocatalyst part 490 will be derived from those of the photocatalyst part described according to the previous embodiments. The photocatalyst part 490 may be disposed on at least one of surfaces of the inner case 412, the plurality of shelves 430, or the plurality of baskets 440.

A light source 215 that irradiates light having a visible-ray wavelength may be disposed in the case 410. For example, the light source 215 may be disposed on the inner case 412. A plurality the light source 215 may be provided. The plurality of light sources 215 may be disposed to be spaced apart from each other. The plurality of light sources 215 may be disposed on sides of spaces partitioned by the plurality of shelves 430, respectively.

As described above, the photocatalyst part may be disposed in the case of the refrigerator. As the light source irradiates visible rays onto the photocatalyst part, generation of pollution materials or propagation of microorganisms that may occur under wet conditions within the refrigerator may be prevented.

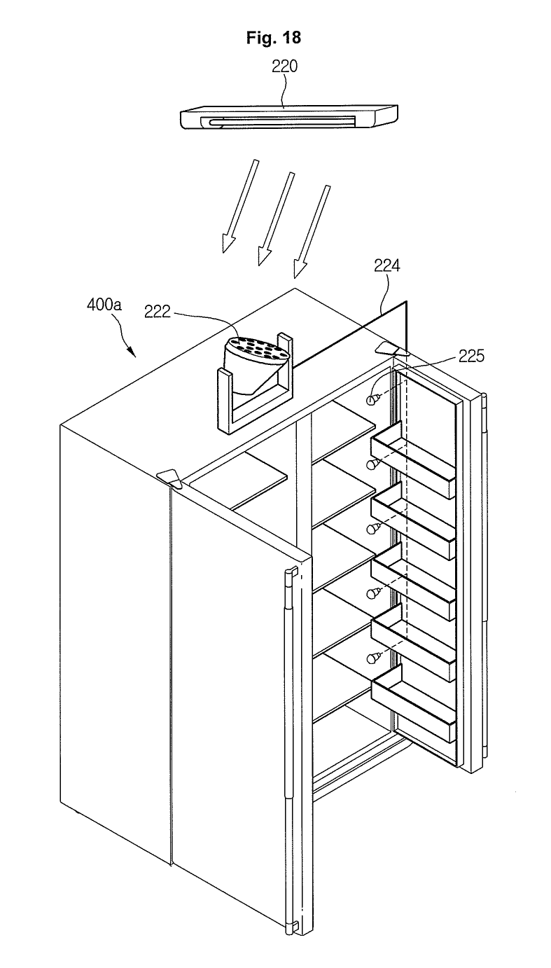

FIG. 18 is a view of a home appliance according to an additional embodiment. Referring to FIG. 18, a refrigerator 400a according to this embodiment may include light source 225 disposed in case 410 and guide device 222 and 224 to guide external light emitted from external light source 222 toward the light source 225.

The guide device 222 and 224 may include light collection part 222 that collects light (visible rays) emitted from the external light source 220, and optical cable 224 to guide the light collected by the light collection part 222 into the case 410. The light collection part 222 may be disposed in the case 410 or disposed at a position spaced apart from the case 110. For example, the light collection part 222 may be disposed on a top surface of the case 410.

The optical cable 224 may extend to the inside of the case 410, and then, may be coupled to the light source 225. The light source 225 may irradiate the visible rays transmitted through the optical cable 224 into the case 410. For example, the light source 225 may include a wire-type light source using an LED or optical fiber.

Another embodiment will be proposed. Although the air conditioner, the dehumidifier, and the refrigerator are exemplified as examples of kind of home appliances in the embodiments discussed above, embodiments are not limited thereto. For example, other home appliances, for example, a washing machine or a dishwasher may be equally applied to the embodiments.

The washing machine may include a tub, in which washing water may be stored, and a drum, in which clothes may be received. The tub and drum may be disposed in a case of the washing machine. The dishwasher may include a tub that defines an accommodation space for dishes, and a sump, in which washing water may be stored. The tub and sump may be disposed in a case of the dishwasher.

A photocatalyst part or photocatalyst may be disposed on a surface of each of inner and outer cases of the washing machine and the dishwasher or a surface of the internal components (the tub, drum, or sump) to irradiate the visible rays onto the photocatalyst part, thereby activating the photocatalyst. Thus, propagation of microorganisms may be restrained to realize antibacterial and deodorization effects.

According to embodiments disclosed herein, as the photocatalyst part, which is an eco-friendly material and reacts with the visible rays, may be provided in the home appliances, various harmful materials may be decomposed, and antibacterial and sterilization functions may be performed. In particular, the photocatalyst may be disposed on parts or portions on which the pollution material may be generated, such as the inside or outside of the case of the home appliance, the heat exchanger, the fan, and the drain device or drain, for example, to prevent the home appliance from be polluted.

Silver phosphate (Ag3PO4), titanium dioxide (TiO2), and an inorganic binder may be mixed with the photocatalyst at a predetermined ratio to maximize efficiency in the viable-ray wavelength region.

The guide hole to guide the external light may be defined in the case of the home appliance, and natural light or light (the external light) emitted from lighting disposed in a building may be introduced into the home appliance. Thus, it may be unnecessary to provide a separate light source for the photocatalyst reaction. The case of the home appliance may be formed of a transparent material to allow the natural light or external light to be introduced into the home appliance through the case, thereby easily realizing the reaction of the photocatalyst part.

Also, as a light source or lighting that irradiates light having a visible-ray wavelength may be disposed in the home appliance, manufacturing costs may be relatively inexpensive when compared to UV lighting, and a life cycle of the lighting may be relatively long.

Embodiments disclosed herein provide a home appliance including a photocatalyst for visible rays, capable of reducing occurrence of pollution materials.

Embodiments disclosed herein provide a home appliance including a photocatalyst for visible rays that may include a case that defines an exterior; and a photocatalyst part or photocatalyst disposed on an inner or outer surface of the case, the photocatalyst part reacting or being activated by light having a visible-ray wavelength. Silver phosphate (Ag3PO4) and titanium dioxide (TiO2) may be mixed to a set weight part ratio.

The case may include at least one hole to introduce external light emitted from outside of the home appliance into the case. A light guide part or guide to guide external light emitted from the outside of the home appliance into the case may be disposed in the case, and the light guide part may be formed of a light-transmissive transparent material.

Light having a visible-ray wavelength may be irradiated through a light source disposed in the case. The light source may include a wire-type lighting source using a light emitting diode (LED) or optical fiber.

The home appliance may further include a light collection part or portion that collects external light emitted from the outside of the home appliance, and an optical cable that guides the light collected by the light collection part into the light source.

The photocatalyst part may be disposed on an outer surface or inner surface of the case.

Silver phosphate (Ag3PO4) and titanium dioxide (TiO2) may be dispersed into a predetermined solvent and applied in the form of a solution to form the photocatalyst part.

The home appliance may be an air conditioner including a heat exchanger, a fan, a filter, and a drain part or drain. The photocatalyst part may be disposed on at least one of surfaces of the heat exchanger, the fan, the filter, or the drain part.

The home appliance may be a dehumidifier including a heat exchanger, a fan, and a drain part or drain. The photocatalyst part may be disposed on at least one of surfaces of the heat exchanger, the fan, or the drain part.

The home appliance may be a refrigerator including an outer case and an inner case, which form the case, one or more shelf to accommodate food, and one or more basket. The photocatalyst part may be disposed on at least one of surfaces of the inner case, the shelf, or the basket.

The home appliance may be a washing machine including a tub, in which washing water may be stored, and a drum, in which clothes may be received. The photocatalyst part may be disposed on at least one of surfaces of the tub or drum.

The home appliance may be a dishwasher including a tub that defines an accommodation space for dishes, and a sump, in which washing water may be stored. The photocatalyst part may be disposed on at least one of surfaces of the tub or the sump.

The photocatalyst part may further include an inorganic binder containing a polysilicate compound. The photocatalyst part may contain about 20 parts to about 50 parts by weight of silver phosphate (Ag3PO4), about 5 parts to about 40 parts by weight of titanium dioxide (TiO2), and about 10 parts to about 40 parts by weight of inorganic binder.

Embodiments disclosed herein provide a home appliance including a photocatalyst for visible rays that may include a case, and a photocatalyst disposed on inner or outer surfaces of the case. The photocatalyst may contain about 20 parts to about 50 parts by weight of silver phosphate (Ag3PO4), about 5 parts to about 40 parts by weight of titanium dioxide (TiO2), and about 10 parts to about 40 parts by weight of inorganic binder and reacts or is activated by visible rays.

A heat exchanger, a fan, a filter, and a drain part or drain may be disposed in the case. The photocatalyst may be disposed on at least one of surfaces of the heat exchanger, the fan, the filter, or the drain part.

At least one guide device or guide to introduce external light emitted from the outside of the home appliance into the case may be disposed in the case, and the guide device may have a guide groove or guide hole. A cover member formed of a transparent material may be disposed on the guide groove.

A light guide part or guide to guide external light emitted from the outside of the home appliance into the case may be disposed in the case. The light guide part may be formed of a light-transmissive transparent material.

The details of one or more embodiments are set forth in the accompanying drawings and the description below. Other features will be apparent from the description and drawings, and from the claims.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

Any reference in this specification to “one embodiment,” “an embodiment,” “example embodiment,” etc., means that a particular feature, structure, or characteristic described in connection with the embodiment is included in at least one embodiment. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A home appliance including a photocatalyst for visible rays, the home appliance comprising:
a case that defines an exterior of the home appliance; and
a photocatalyst disposed inside or outside of the case, the photocatalyst being activated by light having a visible-ray wavelength, wherein the photocatalyst comprises silver phosphate (Ag₃PO₄) and titanium dioxide (TiO₂) mixed to a set weight ratio.

2. The home appliance according to claim 1, wherein the case comprises at least one hole to introduce external light from outside of the home appliance into the case.

3. The home appliance according to claim 1, wherein the case comprises a light guide that guides external light from outside of the home appliance into the case, and wherein the light guide is formed of a transparent material.

4. The home appliance according to claim 1, wherein the light having the visible-ray wavelength is irradiated through a light source disposed within the case.

5. The home appliance according to claim 4, wherein the light source comprises a wire-type light source using a light emitting diode (LED) or optical fiber.

6. The home appliance according to claim 4, further comprising:
a light collection portion that collects external light from outside of the home appliance; and
an optical cable that guides the light collected by the light collection portion into the light source.

7. The home appliance according to claim 1, wherein the photocatalyst is disposed on an outer surface or inner surface of the case.

8. The home appliance according to claim 1, wherein the silver phosphate (Ag₃PO₄) and the titanium dioxide (TiO₂) are dispersed into a predetermined solvent and applied in the form of a solution to form the photocatalyst.

9. The home appliance according to claim 1, wherein the home appliance is an air conditioner comprising a heat exchanger, a fan, a filter, and a drain, and wherein the photocatalyst is disposed on at least one of surfaces of the heat exchanger, the fan, the filter, or the drain.

10. The home appliance according to claim 1, wherein the home appliance is a dehumidifier comprising a heat exchanger, a fan, and a drain, and wherein the photocatalyst is disposed on at least one of surfaces of the heat exchanger, the fan, or the drain.

11. The home appliance according to claim 1, wherein the home appliance is a refrigerator comprising an outer case and an inner case which form the case, at least one shelf to accommodate food, and at least one basket, and wherein the photocatalyst is disposed on at least one of surfaces of the inner case, the shelf, or the basket.

12. The home appliance according to claim 1, wherein the home appliance is a washing machine comprising a tub, in which washing water is stored, and a drum, in which items to be washed are received, and wherein the photocatalyst is disposed on at least one of surfaces of the tub or drum.

13. The home appliance according to claim 1, wherein the home appliance is a dishwasher comprising a tub that defines an accommodation space for dishes, and a sump, in which washing water is stored, and wherein the photocatalyst is disposed on at least one of surfaces of the tub or the sump.

14. The home appliance according to claim 1, wherein the photocatalyst further comprises an inorganic binder containing a polysilicate compound.

15. The home appliance according to claim 14, wherein the photocatalyst contains about 20 parts to about 50 parts by weight of silver phosphate (Ag₃PO₄), about 5 parts to about 40 parts by weight of titanium dioxide (TiO₂), and about 10 parts to about 40 parts by weight of inorganic binder.

16. A home appliance including a photocatalyst for visible rays, the home appliance comprising:
a case; and
a photocatalyst disposed on inner or outer surfaces of the case, wherein the photocatalyst contains about 20 parts to about 50 parts by weight of silver phosphate (Ag3PO4), about 5 parts to about 40 parts by weight of titanium dioxide (TiO2), and about 10 parts to about 40 parts by weight of inorganic binder and is activated by visible rays.

17. The home appliance according to claim 16, wherein at least one of a heat exchanger, a fan, a filter, or a drain are provided in the case, and wherein the photocatalyst is disposed on at least one of surfaces of the heat exchanger, the fan, the filter, or the drain so provided.

18. The home appliance according to claim 16, wherein at least one guide to introduce external light from outside of the home appliance into the case is disposed in the case, and wherein the at least one guide comprises a guide groove or a guide hole.

19. The home appliance according to claim 18, wherein a cover formed of a transparent material is disposed on the guide groove.

20. The home appliance according to claim 16, wherein a light guide to guide external light from outside of the home appliance into the case is disposed in the case, and wherein the light guide is formed of a light-transmissive transparent material.

21. A home appliance including a photocatalyst for visible rays, the home appliance comprising:
a case that defines an exterior of the home appliance;
a plurality of components disposed within the case; and
a photocatalyst disposed on at least one of inner or outer surfaces of the case or surfaces of one of the plurality of components of the case, the photocatalyst being activated by light having a visible-ray wavelength.

22. The home appliance according to claim 21, wherein the photocatalyst comprises silver phosphate (Ag₃PO₄) and titanium dioxide (TiO₂) mixed to a set weight ratio.

23. The home appliance according to claim 22, wherein the silver phosphate (Ag₃PO₄) and the titanium dioxide (TiO₂) are dispersed into a predetermined solvent and applied in the form of a solution to form the photocatalyst.

24. The home appliance according to claim 21, wherein the photocatalyst contains about 20 parts to about 50 parts by weight of silver phosphate (Ag₃PO₄), about 5 parts to about 40 parts by weight of titanium dioxide (TiO₂), and about 10 parts to about 40 parts by weight of inorganic binder and is activated by visible rays.

25. The home appliance according to claim 21, wherein the case comprises at least one hole to introduce external light from outside of the home appliance into the case.

26. The home appliance according to claim 21, wherein the photocatalyst further comprises an inorganic binder containing a polysilicate compound.

27. The home appliance according to claim 21, wherein at least one of a heat exchanger, a fan, a filter, or a drain are provided in the case, and wherein the photocatalyst is disposed on at least one of surfaces of the heat exchanger, the fan, the filter, or the drain so provided.
